(19) United States
(12) Patent Application Publication
(10) Pub. No.: US 2024/0226461 A1
(43) Pub. Date: Jul. 11, 2024

(54) Ophthalmic Nebulizing Delivery System

(71) Applicants: Andrew Pittner, Virginia Beach, VA (US); Stephane Gobron, San Gabriel, CA (US)

(72) Inventors: Andrew Pittner, Virginia Beach, VA (US); Stephane Gobron, San Gabriel, CA (US)

(21) Appl. No.: 18/151,549

(22) Filed: Jan. 9, 2023

Related U.S. Application Data
(60) Provisional application No. 63/403,209, filed on Sep. 1, 2022.
(60) Provisional application No. 63/297,862, filed on Jan. 10, 2022.

(51) Int. Cl. A61M 11/00

(52) U.S. Cl. CPC A61M 11/00; A61M 2210/0612

(57) ABSTRACT

An ophthalmic atomizing delivery system is provided, comprising: a) a frame configured to rest on a user's face, wherein the frame comprises one or more surfaces configured to form a chamber in front of the user's eyes, and wherein the chamber comprises an inner surface opposite the user's eyes; b) a disposable pod comprising a pierceable membrane and a nebulizable liquid; c) a piercing member integrated into the frame and configured to pierce the pierceable membrane and enable the nebulizable liquid to be gravity-fed into a conduit integrated into the frame; and d) a nebulizing system integrated into the frame, wherein the conduit is configured to direct the nebulizable liquid to the nebulizing system, and wherein the nebulizing system is configured to produce a mist from the nebulizable liquid that is directed toward the inner surface of the chamber.

### CROSS-REFERENCE TO RELATED APPLICATIONS

The present application is a U.S. Non-Provisional Utility Patent Application that claims the benefit of U.S. Provisional Patent Application No. 63/403,209, entitled “Ophthalmic Atomizing Delivery System,” filed Sep. 1, 2022 and U.S. Provisional Patent Application No. 63/297,862, entitled “Eyeglasses Vapor Delivering Eye Drop System” filed Jan. 10, 2022; the entire disclosures of which are incorporated herein by reference.

### BACKGROUND OF THE INVENTION

Eyedrops are the main mode of applying medications and moisturizing solutions to the ocular surface. However, this means of ophthalmic delivery has numerous problems. For example, the average eye drop (approximately 50 micro liters) far exceeds the eye's capacity (7 micro liters in the pre-corneal tear film and a maximum of about 30 micro liters in the lower cul-de-sac), which effectively destabilizes and strips the natural tear film. The result is a brief period of massive over-dosage that is then quickly cleared by reflex lacrimation, blinking, and nasolacrimal drainage. Ultimately, sub-therapeutic drug levels are achieved.

Regular use of eye drops also results in local irritations and toxicities that vary widely depending on the pharmacologic agent, preservatives, and other additives being used. Chronic eye drop use for conditions like glaucoma and prolonged infections can cause substantial morbidity. Serious and even fatal reactions have occurred as a result of systemic absorption of eye drops containing sympathomimetic and beta-adrenergic blocking agents via nasolacrimal drainage.

In addition, patients often experience significant difficulties with the mechanics of eye drop administration. Eyedrops are typically applied by the user in a cumbersome and challenging fashion: the user must tilt their head, keep their eyelid open with one hand, and finally must aim and squeeze a bottle/dose to deliver the eyedrops. This process is especially challenging for older patients as it requires flexibility, dexterity, and coordination, which might be reduced with age.

It is estimated that there are 25 million eye drop users in the U.S. alone, and the ophthalmic literature is replete with references to the need for better means of ophthalmic drug delivery. Multiple devices have been attempted, however none have adequately addressed issues such as proper dosing volumes, sterility, and ease of use. Accordingly, there is a need in the art for improved systems and methods for applying medications and moisturizing solutions to the ocular surface.

### SUMMARY OF THE INVENTION

To address the foregoing problems, in whole or in part, and/or other problems that may have been observed by persons skilled in the art, the present disclosure provides compositions and methods as described by way of example as set forth below.

An ophthalmic atomizing delivery system is provided, comprising:
a. a frame configured to rest on a user's face, wherein the frame comprises one or more surfaces configured to form a chamber in front of the user's eyes, and wherein the chamber comprises an inner surface opposite the user's eyes;
b. a disposable pod comprising a pierceable membrane and a nebulizable liquid;
c. a piercing member integrated into the frame and configured to pierce the pierceable membrane and enable the nebulizable liquid to be gravity-fed into a conduit integrated into the frame; and
d. a nebulizing system integrated into the frame, wherein the conduit is configured to direct the nebulizable liquid to the nebulizing system, and wherein the nebulizing system is configured to produce a mist from the nebulizable liquid that is directed toward the inner surface of the chamber.

In some aspects, the inner surface of the chamber is configured to redirect the mist toward the user's eyes and toward an edge of the frame, whereby a vortex of mist is created. In some aspects, the vortex of mist is oriented in a horizontal plane. In other aspects, the vortex of mist is oriented in a vertical plane. In other aspects, the vortex of mist is a 3-dimensional vortex.

In some aspects, the piercing member comprises a piezo and electronic driving system. In other aspects, the piercing member comprises an inkjet head and electronic driving system.

In some aspects, the nebulizable liquid comprises a medication.

In some aspects, the nebulizing system is further configured to produce a heated mist such that a sauna effect is produced in the chamber.

In some aspects, the pierceable membrane is oriented 90°±25° in relation to the ground. In other aspects, the pierceable membrane is oriented 90°±15° in relation to the ground.

In some aspects, the frame comprises a second piercing member integrated into the frame and configured to pierce the pierceable membrane to break a vacuum in the pod.

In another embodiment, an ophthalmic atomizing delivery system is provided, comprising:
a. a frame configured to rest on a user's face, wherein the frame comprises one or more surfaces configured to form a chamber in front of the user's eyes, and wherein the chamber comprises an inner surface opposite the user's eyes;
b. a disposable pod comprising a vertically-oriented pierceable membrane and a nebulizable liquid;
c. a piercing member integrated into the frame and configured to pierce the pierceable membrane and enable the nebulizable liquid to be gravity-fed into a conduit integrated into the frame;
d. a second piercing member integrated into the frame and configured to pierce the pierceable membrane to break a vacuum in the pod;
e. a nebulizing system integrated into the frame, wherein the conduit is configured to direct the nebulizable liquid to the nebulizing system, and wherein the nebulizing system is configured to produce a mist from the nebulizable liquid that is directed toward the inner surface of the chamber, wherein the inner surface of the chamber is configured to redirect the mist toward the user's eyes and toward an edge of the frame, whereby a vortex of mist is created, and wherein the vortex of mist is a 3-dimensional vortex.

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF DRAWINGS

Having thus described the subject matter of the present invention in general terms, reference will now be made to the accompanying drawings, which are not necessarily drawn to scale.

FIG. 1 shows a perspective view of an ophthalmic atomizing delivery system according to an embodiment of the present invention.

FIG. 2 shows an exploded perspective view of the ophthalmic atomizing delivery system of FIG. 1.

FIG. 3 shows a cross-section of a perspective view of the ophthalmic atomizing delivery system of FIG. 1.

FIG. 4 shows a cross-section of a top view of the ophthalmic atomizing delivery system of FIG. 1.

FIG. 5 shows a cut-away view of the nebulizing system of the ophthalmic atomizing delivery system of FIG. 1.

FIG. 6A shows a disposable pod container; while FIG. 6B shows the disposable pod comprising a nebulizable liquid with a vertically oriented pierceable membrane.

FIG. 7 shows the disposable pod of FIG. 6 loaded into the ophthalmic atomizing delivery system of FIG. 1 and ready to be pierced by the piercing member.

FIG. 8 shows the disposable pod of FIG. 6 after use, showing a clean piercing from the piercing member.

FIGS. 9A-9D show different lens shapes for use within the ophthalmic atomizing delivery system: FIG. 9A shows a left shaped lens front view; FIG. 9B shows a perspective view of the shaped lens; FIG. 9C shows a vertical X-section of the left shaped lens; and FIG. 9D shows a horizontal X-section of the left shaped lens.

### DETAILED DESCRIPTION OF THE INVENTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. Therefore, it is to be understood that the subject matter of the present invention is not to be limited to the specific embodiments disclosed and that modifications and other embodiments are intended to be included within the scope of the appended claims.

### Ophthalmic Nebulizing Delivery System

The present invention relates to an ophthalmic atomizing delivery system that improves the ease of delivering eyedrop solutions and optimizes moisturization of the corneal surfaces of a user's eyes. A liquid solution is delivered as a gentle mist to create an optimal moisturizing environment for the eyes. By design, the system delivers the mist not straight onto the eye but onto an inner surface of the frame/chamber. Accordingly, the user does not have to endure a stream of fluid directed into the eyes, which increases comfort while decreasing the blink reflex that impedes eyedrop administration. Furthermore, the mist remains in contact with the corneal surface for a longer period of time than standard eye drops, further increasing the moisturizing efficacy of the system.

Accordingly, the present invention relates to an ophthalmic atomizing delivery system, comprising:
a) a frame configured to rest on a user's face, wherein the frame comprises one or more surfaces configured to form a chamber in front of the user's eyes, and wherein the chamber comprises an inner surface opposite the user's eyes;
b) a disposable pod comprising a vertically oriented pierceable membrane and a nebulizable liquid;
c) a piercing member integrated into the frame and configured to pierce the pierceable membrane and enable the nebulizable liquid to be gravity-fed into a conduit integrated into the frame; and
d) a nebulizing system integrated into the frame, wherein the conduit is configured to direct the nebulizable liquid to the nebulizing system, and wherein the nebulizing system is configured to produce a mist from the nebulizable liquid that is directed toward the inner surface of the chamber.

In some embodiments, the nebulizing system comprises a piezo nebulizer, an electronic board to drive the piezo component, and a battery to power the system.

The nebulizable liquid, e.g., a liquid solution, is gravity-fed to the nebulizing system, and the resulting mist is directed into the chamber. The nebulizable liquid may be packaged in single-dose foil-sealed pods that are inserted into the system and pushed against the piercing member that connects the nebulizable liquid to the conduit. The piercing member is designed to minimize or eliminate leakage (as the pierceable membrane, e.g., foil, is positioned vertically).

Active components to generate mist can be a piezo flat donut vibrating a metal disc that includes a plurality of holes in the center portion of the disc. The piezo vibration amplitude and frequency is controlled by a circuit board and powered by a low voltage DC battery. Alternatively, an inkjet printer head or an array of heads can be used instead of a piezo, whereby the inkjet head(s) is/are controlled by a circuit board and powered by a low voltage DC battery.

The disposable pod is designed similarly to a small individual coffee creamer: a small plastic container with a pierceable membrane, e.g., a foil-sealed surface, to keep the liquid contained. The pierceable membrane can be made of a plastic film, an aluminum foil, or a metal foil laminated with a thin plastic layer. Foil is typically heat-sealed against the flange of the container. Optionally, a peripheral protruding rim can be included on the flange, just around the edge of the cavity to provide with a small thin plastic mass that can easily be melted during the sealing operation.

Alternatively, the disposable pod could also be a small individual dose similar to commercially available eyedrop doses: a small plastic container is formed through a blow-fill-seal process and aseptically filed with the solution. The piercing feature could be an over molded needle tip. An optional thread could be included on the pod container neck to help with centering and to generate greater piercing forces with ease.

In some embodiments, the frame is shaped like a pair of goggles or eyeglasses that sit on the face of the user to create a chamber in front of the user's eyes.

An innovative feature of this product is the fact that the mist can be delivered in a way to generate a small, gentle vortex around the corneal surface to help with the moisturizing efficacy of the system. The generated mist stream is oriented into the chamber at a specific angle and velocity. By defining the correct angle of approach (with respect to the optical axis and the frame), and having the proper mist velocity, the system can be tuned to generate a small vortex of mist to continuously moisturize the corneal surface, thereby optimizing the moisturizing efficacy of the system.

Accordingly, in some aspects, the inner surface of the chamber is configured to redirect the mist toward the user's eyes and toward an edge of the frame, whereby a vortex of mist is created. In some aspects, the vortex of mist is oriented in a horizontal plane. In other aspects, the vortex of mist is oriented in a vertical plane. In other aspects, the vortex of mist is a 3-dimensional vortex.

Another innovative feature is the use of lenses shaped to foster the creation of the vortex. The lens is designed to reorient the mist towards the corneal surface to generate a small vortex. Having a small angle of approach, where the flow is almost parallel to the surface for a short distance, the lens shape redirects the flow towards the eye and/or a corner of the chamber to create a vortex.

In some aspects, the piercing member comprises a piezo and electronic driving system (comprising, e.g., a battery and an electronic board). In other aspects, the piercing member comprises an inkjet head and electronic driving system. In other aspects, the piercing mechanism is a protruding triangular X-section feature that has a bottom edge and a sharp piercing point (e.g., the front is cut at an angle with respect to the surface to be pierced). In some aspects, the nebulizable liquid comprises a medication.

In some aspects, the nebulizing system is further configured to produce a heated mist such that a sauna effect is produced in the chamber.

In some aspects, the vertically oriented pierceable membrane is oriented 90°±25° in relation to the ground. In other aspects, the vertically oriented pierceable membrane is oriented 90°±15° in relation to the ground.

In some aspects, the frame comprises a second piercing member integrated into the frame and configured to pierce the pierceable membrane to break a vacuum in the pod.

In another embodiment, an ophthalmic atomizing delivery system is provided, comprising:
a. a frame configured to rest on a user's face, wherein the frame comprises one or more surfaces configured to form a chamber in front of the user's eyes, and wherein the chamber comprises an inner surface opposite the user's eyes;
b. a disposable pod comprising a vertically-oriented pierceable membrane and a nebulizable liquid;
c. a piercing member integrated into the frame and configured to pierce the pierceable membrane and enable the nebulizable liquid to be gravity-fed into a conduit integrated into the frame;
d. a second piercing member integrated into the frame and configured to pierce the pierceable membrane to break a vacuum in the pod;
e. a nebulizing system integrated into the frame, wherein the conduit is configured to direct the nebulizable liquid to the nebulizing system, and wherein the nebulizing system is configured to produce a mist from the nebulizable liquid that is directed toward the inner surface of the chamber, wherein the inner surface of the chamber is configured to redirect the mist toward the user's eyes and toward an edge of the frame, whereby a vortex of mist is created, and wherein the vortex of mist is a 3-dimensional vortex.

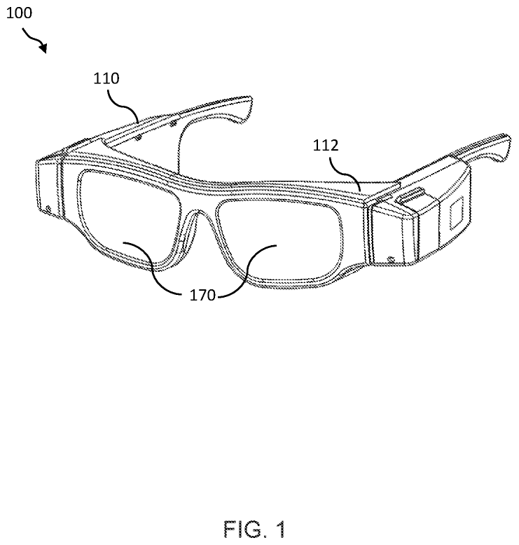

Turning now to FIG. 1, a perspective view is shown of an ophthalmic atomizing delivery system 100 according to an embodiment of the present invention. A goggle-shaped frame 110 configured to rest on a user's face includes one or more surfaces 112 configured to form a chamber in front of the user's eyes. The system 100 also includes lenses 170.

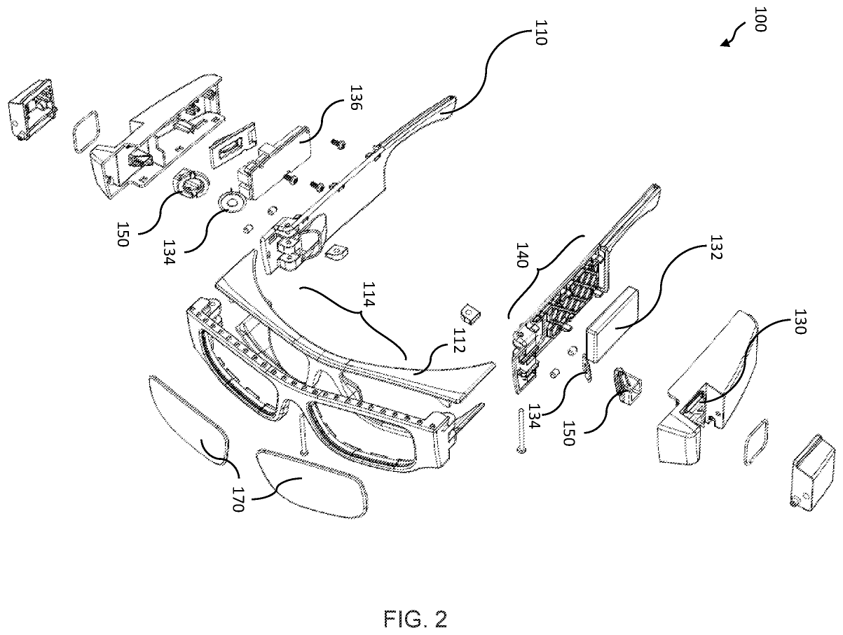

Turning now to FIG. 2, an exploded perspective view of the ophthalmic atomizing delivery system 100 of FIG. 1 is shown. Goggle-shaped frame 110 is configured to rest on a user's face and includes one or more surfaces 112 configured to form a chamber 114 in front of the user's eyes. The system 100 also includes a piercing member 130 integrated into the frame 110, comprising a piezo 134, a battery 132, and an electronic board 136. The system 100 also includes a conduit 140 integrated into the frame 110, as well as a nebulizing system 150 integrated into the frame 110. The system 100 also includes lenses 170.

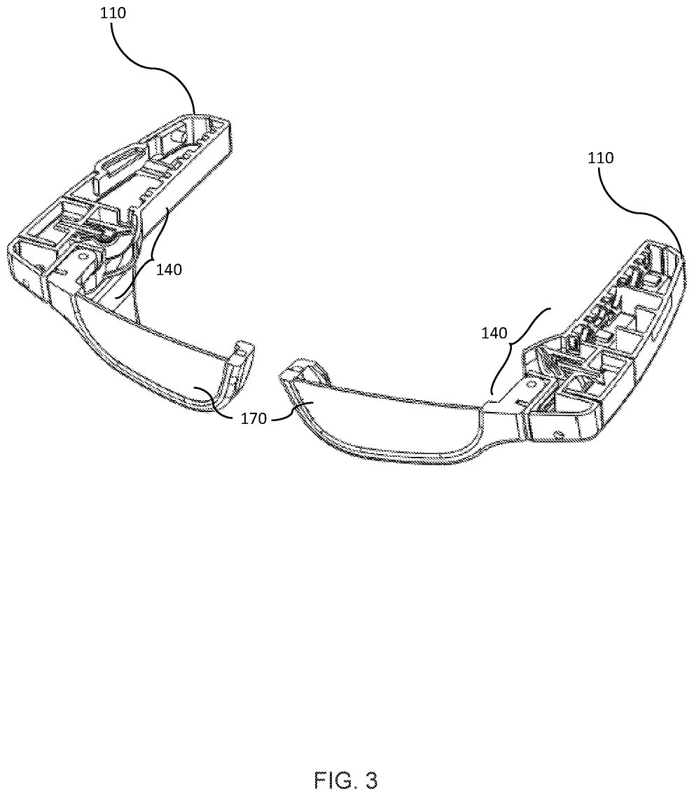

Turning now to FIG. 3, a cross-section of a perspective view of the ophthalmic atomizing delivery system 100 of FIG. 1 is shown, depicting the path of conduit 140 within frame 110 (parts of lenses 170 are also shown).

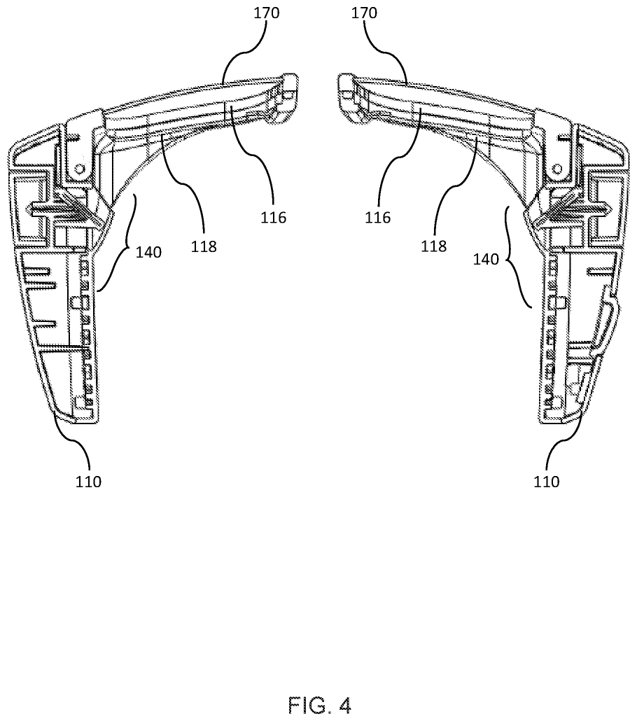

Turning now to FIG. 4, a cross-section of a top view of the ophthalmic atomizing delivery system 100 of FIG. 1 is shown, including conduit 140 within frame 110 (parts of lenses 170 are also shown). Also shown is an inner surface 116 opposite the user's eyes and an edge of the frame 118.

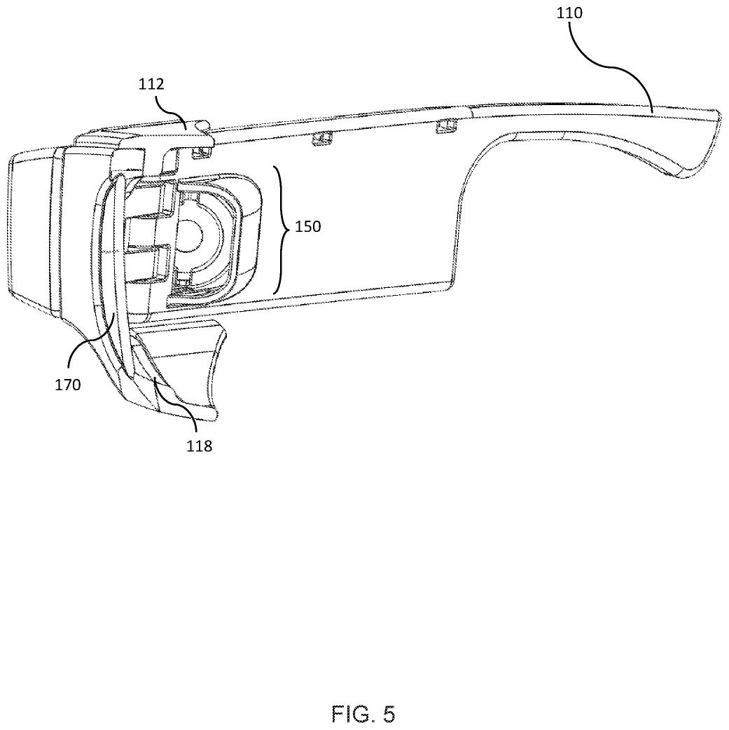

Turning now to FIG. 5, a cut-away view is shown of the nebulizing system 150 of the ophthalmic atomizing delivery system 100 of FIG. 1. Nebulizing system 150 is integrated into frame 110. The one or more surfaces 112, the edge of the frame 118, and part of the lenses 170 are also shown.

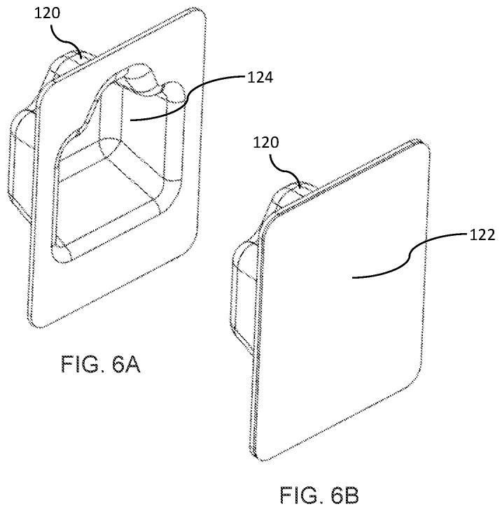

Turning now to FIG. 6A, a disposable pod 120 is shown comprising a nebulizable liquid 124; while FIG. 6B shows the disposable pod 120 with a vertically oriented pierceable membrane 122.

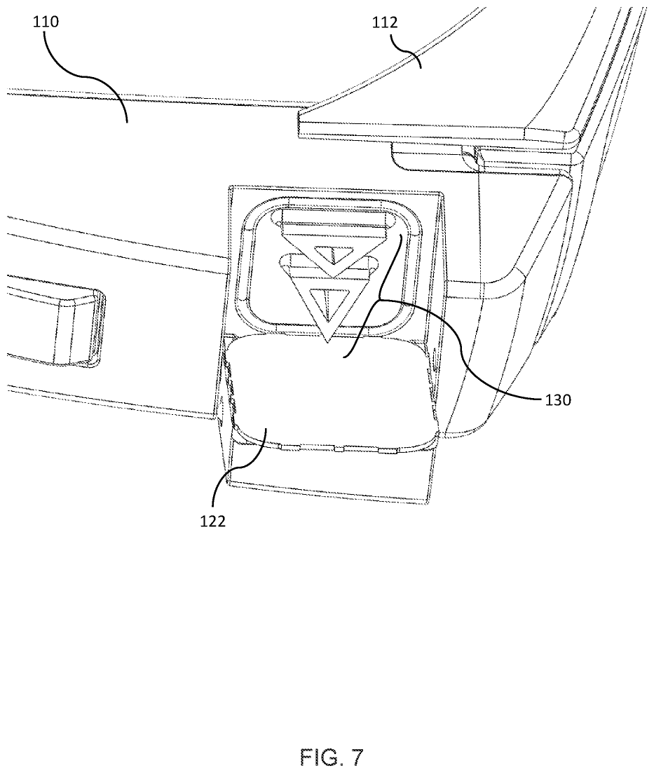

Turning now to FIG. 7, the disposable pod of FIG. 6 is shown loaded into the ophthalmic atomizing delivery system 100 of FIG. 1 and ready for vertically oriented pierceable membrane 122 to be pierced by piercing member 130. Also shown is frame 110 and one or more surfaces 112.

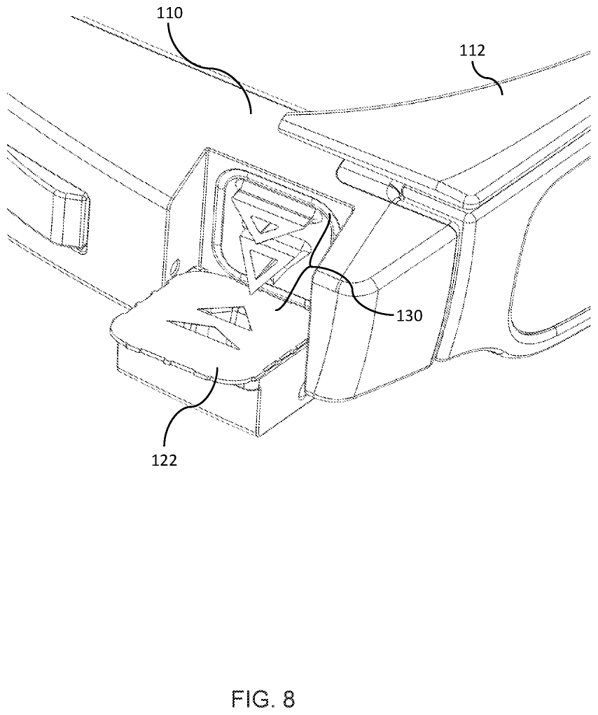

Turning now to FIG. 8, the disposable pod 120 of FIG. 6 is shown after use, showing a clean piercing from piercing member 130 in vertically oriented pierceable membrane 122. Also shown is frame 110 and one or more surfaces 112.

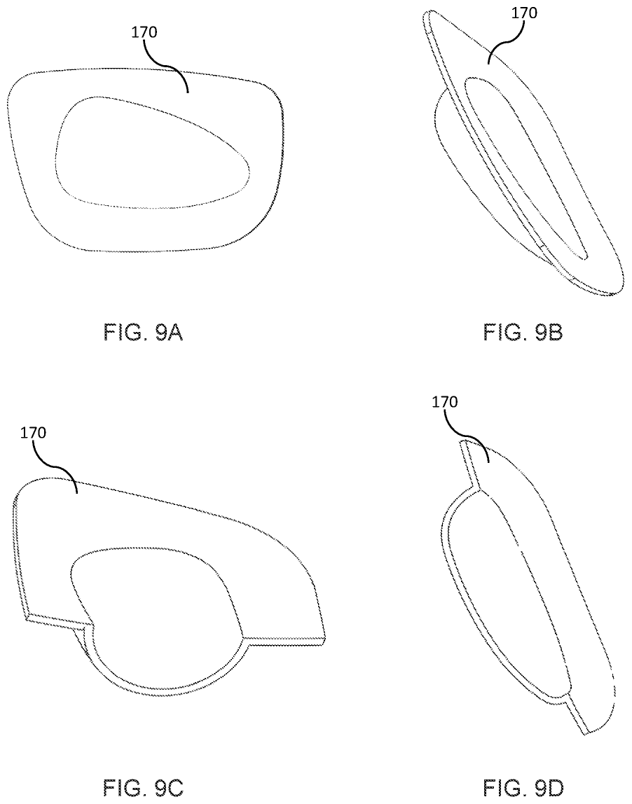

Turning now to FIGS. 9A-9D, different lens 170 shapes are shown for use within the ophthalmic atomizing delivery system: FIG. 9A shows a left shaped lens 170 front view; FIG. 9B shows a perspective view of the shaped lens 170; FIG. 9C shows a vertical X-section of the left shaped lens 170; and FIG. 9D shows a horizontal X-section of the left shaped lens 170.

### General Definitions

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments±100%, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

1. An ophthalmic atomizing delivery system, comprising:
a. a frame configured to rest on a user's face, wherein the frame comprises one or more surfaces configured to form a chamber in front of the user's eyes, and wherein the chamber comprises an inner surface opposite the user's eyes:
b. a disposable pod comprising a pierceable membrane and a nebulizable liquid:
c. a piercing member integrated into the frame and configured to pierce the pierceable membrane and enable the nebulizable liquid to be gravity-fed into a conduit integrated into the frame; and
d. a nebulizing system integrated into the frame, wherein the conduit is configured to direct the nebulizable liquid to the nebulizing system, and wherein the nebulizing system is configured to produce a mist from the nebulizable liquid that is directed toward the inner surface of the chamber.

2. The ophthalmic atomizing delivery system of claim 1, wherein the inner surface of the chamber is configured to redirect the mist toward the user's eyes and toward an edge of the frame, whereby a vortex of mist is created.

3. The ophthalmic atomizing delivery system of claim 2, wherein the vortex of mist is oriented in a horizontal plane.

4. The ophthalmic atomizing delivery system of claim 2, wherein the vortex of mist is oriented in a vertical plane.

5. The ophthalmic atomizing delivery system of claim 2, wherein the vortex of mist is a 3-dimensional vortex.

6. The ophthalmic atomizing delivery system of claim 1, wherein the nebulizing system comprises a piezo and electronic driving system.

7. The ophthalmic atomizing delivery system of claim 1, wherein the nebulizing system comprises an inkjet head and electronic driving system.

8. The ophthalmic atomizing delivery system of claim 1, wherein the nebulizable liquid comprises a medication.

9. The ophthalmic atomizing delivery system of claim 1, wherein the nebulizing system is further configured to produce a heated mist such that a sauna effect is produced in the chamber.

10. The ophthalmic atomizing delivery system of claim 1, wherein the pierceable membrane is oriented 90°±25° in relation to the ground.

11. The ophthalmic atomizing delivery system of claim 1, wherein the pierceable membrane is oriented 90°±15° in relation to the ground.

12. The ophthalmic atomizing delivery system of claim 1, wherein the frame comprises a second piercing member integrated into the frame and configured to pierce the pierceable membrane to break a vacuum in the pod.

13. An ophthalmic atomizing delivery system, comprising:
a. a frame configured to rest on a user's face, wherein the frame comprises one or more surfaces configured to form a chamber in front of the user's eyes, and wherein the chamber comprises an inner surface opposite the user's eyes:
b. a disposable pod comprising a vertically-oriented pierceable membrane and a nebulizable liquid:
c. a piercing member integrated into the frame and configured to pierce the pierceable membrane and enable the nebulizable liquid to be gravity-fed into a conduit integrated into the frame:
d. a second piercing member integrated into the frame and configured to pierce the pierceable membrane to break a vacuum in the pod:
e. a nebulizing system integrated into the frame, wherein the conduit is configured to direct the nebulizable liquid to the nebulizing system, and wherein the nebulizing system is configured to produce a mist from the nebulizable liquid that is directed toward the inner surface of the chamber, wherein the inner surface of the chamber is configured to redirect the mist toward the user's eyes and toward an edge of the frame, whereby a vortex of mist is created, and wherein the vortex of mist is a 3-dimensional vortex.
